(19) United States
(12) Patent Application Publication
(10) Pub. No.: US 2021/0282933 A1
(43) Pub. Date: Sep. 16, 2021

(54) Drug-Eluting Spacer for Joints of the Human Body

(71) Applicant: Exactech, Inc.

(72) Inventors: Andrew Sutherland, Gainesville, FL (US); Wayne Crawford, Gainesville, FL (US)

(73) Assignee: Exactech, Inc., Gainesville, FL (US)

(21) Appl. No.: 16/332,614

(22) PCT Filed: Sep. 12, 2017
(86) PCT No.: PCT/US17/51134

§ 371 Date: Mar. 12, 2019

Related U.S. Application Data
(60) Provisional application No. 62/393,406, filed on Sep. 12, 2016.

(51) Int. Cl. A61F 2/30; A61F 2/38; A61K 31/665; A61K 31/43; A61K 31/65; A61K 31/18; A61K 31/4164; A61K 31/407; A61K 31/545; A61K 31/351; A61K 31/165; A61K 31/505; A61K 45/06; A61K 38/14; A61K 31/7036

(52) U.S. Cl. CPC A61F 2/30724; A61F 2002/30677; A61F 2/389; A61K 31/665; A61K 31/43; A61K 31/65; A61K 31/18; A61K 31/4164; A61K 31/407; A61K 31/545; A61K 31/351; A61K 31/165; A61K 31/505; A61K 45/06; A61K 38/14; A61K 31/7036; A61F 2/3859

(57) ABSTRACT

A drug-eluting spacer for temporary implantation in a knee joint includes a femoral component configured to interface with a femur, a tibial tray component having an upper surface, a lower surface, and a shaft extending from the lower surface, the shaft configured to be positioned axially within a tibia, the lower surface configured to interface with the tibia, and a tibial insert component having an upper surface and a lower surface, the lower surface of the tibial insert component configured to engage the upper surface of the tibial tray component, the upper surface of the tibial insert component configured to receive the femoral component in an articulating manner. The femoral component, the tibial tray component, and the tibial insert component carry joint loads when implanted. The drug-eluting spacer is configured to elute a biologically active agent in an amount effective to treat an infection of the knee joint.

### CROSS-REFERENCE TO RELATED APPLICATION

This is an international (PCT) application relating to and claiming the benefit of commonly-owned, copending U.S. Provisional Patent Application No. 62/393,406, filed Sep. 12, 2016, entitled “DRUG-ELUTING SPACER FOR JOINTS OF THE HUMAN BODY,” the contents of which are incorporated by reference herein in their entirety.

### FIELD

The present invention refers to a drug-eluting spacer for the temporary replacement of joint prostheses that require to be removed for various reasons, such as, for example, due to an infection. Such drug-eluting spacer allows, over the period of time required for treating the joint, preserving the space required for the implantation of a new articular prosthesis and maintaining a good movement of the joint.

### BACKGROUND

Joint prostheses can be subject to removal, for a variety of reasons, such as, for example, local infection of the joint after implantation of the prosthesis. In such cases, the infected prosthesis may not be immediately replaced with a new prosthesis, given that the site of the removed joint replacement is required to be treated using suitable antibiotic medicines. During the period of time required for the antibiotic treatment, preservation of the joint is required for the implantation of a new articular prosthesis, to prevent the tissues from shortening, the joint from being subjected to atrophy and the muscles from losing tonicity.

### SUMMARY

In one embodiment, the present invention provides a drug-eluting spacer for temporary implantation in a knee joint of a patient,

wherein the drug-eluting spacer is configured to elute at least one biologically active agent in an amount effective to treat an infection of the knee joint of the patient,

wherein the drug-eluting spacer comprises:
a) a femoral component configured to interface with a femur of the patient;
b) a tibial tray component, wherein the tibial tray comprises an upper surface and a lower surface, and the lower surface of the tibial tray component is disposed adjacent a tibia of the patient
   wherein the lower surface of the tibial tray component comprises a shaft extending downward from the lower surface, and
   wherein the shaft is adapted to be located axially within the tibia; and
c) a tibial insert component, wherein the tibial insert component comprises an upper surface and a lower surface;
   wherein the upper and lower surfaces are separated by a thickness,
   wherein the lower surface of the tibial insert component is configured to lockingly engage with the upper surface of the tibial tray component,
   wherein the locked tibial insert component and tibial tray component carry joint loads when implanted in the patient, and
   wherein the upper surface of the tibial insert component is configured to receive the femoral component in an articulating manner.

In one embodiment, the drug-eluting spacer further comprises a tibial spacer component, positionable between the tibial tray component and the tibial insert component, wherein the tibial spacer component comprises an upper surface and a lower surface, wherein the upper surface of the tibial spacer component is lockingly engaged with the lower surface of the tibial insert component, and wherein the lower surface of the tibial spacer component is lockingly engaged with the upper surface of the tibial tray component.

In one embodiment, the locked tibial insert component/tibial spacer component and tibial tray component carry joint loads when implanted in the patient.

In one embodiment, the femoral component has an anterior side and a posterior side, the femoral component including a pair of laterally spaced condylar portions, each of which has a surface which is configured to match generally the lateral profile of an anatomical femoral condyle.

In one embodiment, the present invention provides a kit to form a drug-eluting spacer for temporary implantation in a knee joint of a patient,

wherein the drug-eluting spacer is configured to elute at least one biologically active agent in an amount effective to treat an infection of the knee joint of the patient,

wherein the kit comprises:
a) a femoral component configured to interface with a femur of the patient;
b) a tibial tray component, wherein the tibial tray component comprises an upper surface and a lower surface, and the lower surface of the tibial tray component is disposed adjacent a tibia of the patient
   wherein the lower surface of the tibial tray component comprises a shaft extending downward from the lower surface, and
   wherein the shaft is adapted to be located axially within the tibia;
c) a plurality of tibial insert components of a first size, wherein each individual tibial insert component within the plurality comprises an upper surface and a lower surface;
   wherein the upper and lower surfaces are separated by a thickness,
   wherein each individual tibial insert component within the plurality has a different thickness from any other individual tibial insert components within the plurality,
   wherein the lower surface of the tibial insert component is configured to lockingly engage with the upper surface of the tibial tray component,
   wherein the locked tibial spacer component and tibial tray component carry joint loads when implanted in the patient, and
   wherein the upper surface of the tibial insert component is configured to receive the femoral component in an articulating manner; and
d) a plurality of tibial insert components of a at least one additional size, wherein each individual tibial insert component within the plurality comprises an upper surface and a lower surface;
   wherein the upper and lower surfaces are separated by a thickness,
   wherein each individual tibial insert component within the plurality has a different thickness from any other individual tibial insert components within the plurality,
   wherein the lower surface of the tibial insert component is configured to lockingly engage with the upper surface of the tibial tray component,
   wherein the locked tibial spacer component and tibial tray component carry joint loads when implanted in the patient, and
   wherein the upper surface of the tibial insert component is configured to receive the femoral component in an articulating manner.

In one embodiment, the drug-eluting spacer further comprises a tibial spacer component, positionable between the tibial tray component and the tibial insert component, wherein the tibial spacer component comprises an upper surface and a lower surface, wherein the upper surface of the tibial spacer component is lockingly engaged with the lower surface of the tibial insert component, and wherein the lower surface of the tibial spacer component is lockingly engaged with the upper surface of the tibial tray component.

In one embodiment, the locked tibial insert component/tibial spacer component and tibial tray component carry joint loads when implanted in the patient.

In one embodiment, the size of the tibial insert components of the first size are the same as the size of the tibial tray component.

In one embodiment, the size of the tibial insert components of the at least one additional size is larger than the size of the tibial insert components of the first size. Alternatively, the size of the tibial insert components of the at least one additional size are smaller than the size of the tibial insert components of the first size.

In one embodiment, the femoral component has an anterior side and a posterior side, the femoral component including a pair of laterally spaced condylar portions, each of which has a surface which is configured to match generally the lateral profile of an anatomical femoral condyle.

In an embodiment, a drug-eluting spacer for temporary implantation in a knee joint of a patient includes a femoral component, a tibial tray component, and a tibial insert component, the femoral component configured to interface with a femur of the patient, the tibial tray component having an upper surface, a lower surface opposite the upper surface, and a shaft extending from the lower surface, the shaft configured to be positioned axially within a tibia of the patient, the lower surface configured to interface with the tibia of the patient, the tibial insert component having an upper surface and a lower surface opposite the upper surface, the lower surface of the tibial insert component configured to lockingly engage the upper surface of the tibial tray component, the upper surface of the tibial insert component configured to receive the femoral component in an articulating manner, wherein the femoral component, the tibial tray component, and the tibial insert component carry joint loads when implanted in the patient, and wherein the drug-eluting spacer is configured to elute at least one biologically active agent in an amount effective to treat an infection of the knee joint of the patient.

In an embodiment, the at least one biologically active agent includes at least one antibiotic. In an embodiment, the at least one antibiotic includes at least one of an aminoglycoside, an ansamycin, a carbapenem, a cephalosporin, a glycopeptide, a lincosamide, a macrolide, a monobactam, a penicillin, a penicillin combination, a polypeptide, a quinolone, a sulfonamide, a tetracycline, a drug against mycobacteria, arsphenamine, chloramphenicol, fosfomycin, fusidic acid, linezolid, metronidazole, mupirocin, platensimycin, quinupristin/dalfopristin, rifaximin, thiamphenicol, tigecycline, imidazole, trimethoprim, or combinations thereof. In an embodiment, the at least one antibiotic includes at least one of vancomycin, gentamicin, or combinations thereof. In an embodiment, the at least one antibiotic includes at least one of vancomycin at a concentration of between 2.5% and 20% by weight, gentamicin at a concentration of between 2.5% and 20% by weight, or combinations thereof.

In an embodiment, the at least one biologically active agent includes at least one antifungal agent. In an embodiment, the at least one antifungal agent includes at least one of an azole, an echinocandin, a polyene, or combinations thereof

In an embodiment, the tibial tray component is made from a spacer material including a structural material and the at least one biologically active agent. In an embodiment, the structural material includes at least one of bone cement, a polymer, a biodegradable polymer, a biocompatible polymer, a bioabsorbable polymer, or combinations thereof In an embodiment, the at least one biologically active agent comprises about 20% or less of the spacer material by weight. In an embodiment, the at least one biologically active agent is at least one of embedded into the structural material, impregnated into the structural material, or coated onto the structural material.

In an embodiment, the tibial tray component includes a projection projecting from the upper surface thereof, the tibial insert component includes a recess formed within the lower surface thereof, and the projection and the recess cooperate to lockingly engage the tibial insert component to the tibial tray component when the lower surface of the tibial insert component abuts the upper surface of the tibial tray component.

In an embodiment, the tibial insert component is made from a spacer material including a structural material and the at least one biologically active agent. In an embodiment, the femoral component is made from a spacer material including a structural material and the at least one biologically active agent.

In an embodiment, the shaft of the tibial tray component has a diameter in a range between 5 mm and 25 mm and a length in a range between 5 mm and 175 mm.

In an embodiment, a kit to form a drug-eluting spacer for temporary implantation in a knee joint of a patient includes a femoral component, a tibial tray component, a first plurality of tibial insert components of a first size, and a second plurality of tibial insert components of a second size, the femoral component configured to interface with a femur of the patient, the tibial tray component having an upper surface, a lower surface opposite the upper surface, and a shaft extending from the lower surface, the shaft configured to be positioned axially within a tibia of the patient, the lower surface configured to interface with the tibia of the patient, each of the first plurality of tibial insert components having an upper surface, a lower surface opposite the upper surface, and a thickness between the upper and lower surfaces, each of the tibial insert components in the first plurality having a different thickness from any other individual tibial insert component within the first plurality of tibial insert components, the lower surface of each of the tibial insert components within the first plurality configured to lockingly engage the upper surface of the tibial tray component, the upper surface of each of the tibial insert components within the first plurality configured to receive the femoral component in an articulating manner, each of the second plurality of tibial insert components having an upper surface, a lower surface opposite the upper surface, and a thickness between the upper and lower surfaces, each of the tibial insert components in the second plurality having a different thickness from any other individual tibial insert component within the second plurality of tibial insert components, the lower surface of each of the tibial insert components within the second plurality configured to lockingly engage the upper surface of the tibial tray component, the upper surface of each of the tibial insert components within the second plurality configured to receive the femoral component in an articulating manner, wherein the femoral component, the tibial tray component, and the tibial insert component carry joint loads when implanted in the patient, and wherein the drug-eluting spacer is configured to elute at least one biologically active agent in an amount effective to treat an infection of the knee joint of the patient.

In an embodiment, the first size is the same as a size of the tibial tray. In an embodiment, the second size is larger than the first size. In an embodiment, the second size is smaller than the first size.

In an embodiment, a drug-eluting spacer for temporary implantation in a joint of a patient includes a first bone component, a second bone tray component, and an insert component, the first bone component configured to interface with a first bone to a first side of the joint, the second bone tray component having a first surface, a second surface opposite the first surface, and a shaft extending from the second surface, the second surface configured to interface with a second bone to a second side of the joint, the shaft configured to be positioned axially within the second bone, the insert component having a first surface and a second surface opposite the first surface, the second surface of the insert component configured to lockingly engage the first surface of the second bone tray component, the first surface of the insert component configured to receive the first bone component in an articulating manner, wherein the first bone component, the second bone tray component, and the insert component carry joint loads when implanted in the patient, and wherein the drug-eluting spacer is configured to elute at least one biologically active agent in an amount effective to treat an infection of the joint of the patient.

### BRIEF DESCRIPTION OF THE DRAWINGS

FIG. 1 shows a top view of a tibial tray component of a spacer according to some embodiments of the present invention.

FIG. 2 shows an anterior/posterior view of a tibial tray component of a spacer according to some embodiments of the present invention.

FIG. 3 shows a bottom view of a tibial tray component of a spacer according to some embodiments of the present invention.

FIG. 4 shows a bottom view of a tibial insert component of a spacer according to some embodiments of the present invention.

FIG. 5 shows a medial/lateral view of a tibial insert component with a tibial tray component of a spacer according to some embodiments of the present invention.

FIG. 6 shows an anterior/posterior view of a tibial insert component engaged with a tibial tray component of a spacer according to some embodiments of the present invention.

FIG. 7 shows a medial/lateral view of a tibial insert component and a tibial tray component of a spacer according to some embodiments of the present invention.

FIG. 8 shows an anterior/posterior view of a tibial insert component and a tibial tray component of a spacer according to some embodiments of the present invention.

FIG. 9 shows a medial/lateral view of a femoral component, a tibial insert component and a tibial tray component of a spacer according to some embodiments of the present invention.

FIG. 10 shows an anterior/posterior view of a femoral component, a tibial insert component and a tibial tray component of a spacer according to some embodiments of the present invention.

FIG. 11 shows a photograph of various sizes of a tibial tray component of a spacer according to some embodiments of the present invention.

### DETAILED DESCRIPTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention. In addition, throughout the specification, the meaning of “a,” “an,” and “the” include plural references. The meaning of “in” includes “in” and “on.” Any ranges described herein are inclusive (i.e., include any upper and lower bounds described).

In some embodiments, the present invention provides a drug-eluting spacer for temporary implantation in a joint of a patient to treat an infection of the joint. In some embodiments, the patient has had an implant removed due to a local infection of the joint, and the drug-eluting spacer treats the local infection.

### The Biological Agent

In some embodiments, the drug-eluting spacer is configured to elute at least one biologically active agent in an amount effective to treat an infection of the joint of the patient.

Exemplary biologically active agents include anti-microbial agents, such as, for example, aminoglycosides (such as, for example, amikacin, gentamicin, kanamycin, neomycin, netilmicin, tobramycin, or paromomycin); ansamycins (such as, for example, geldanamycin, or herbimycin); carbacephem (such as, for example, loracarbef), carbapenems (such as, for example, ertapenem, doripenem, imipenem/cilastatin, or meropenem); cephalosporins (such as, for example, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, or ceftobiprole); glycopeptides (such as, for example, teicoplanin, vancomycin, or telavancin); lincosamides (such as, for example, clindamycin, or lincomycin); macrolides (such as, for example, azithromycin, clarithromycin, dirithromycin, erythromycin, roxithromycin, troleandomycin, telithromycin, spectinomycin, spiramycin); monobactams (such as, for example, aztreonam, nitrofurans, furazolidone, or nitrofurantoin), penicillins or penicillin combinations (such as, for example, amoxicillin, ampicillin, azlocillin, carbenicillin, cloxacillin, dicloxacillin, flucloxacillin, mezlocillin, methicillin, nafcillin, oxacillin, penicillin v, piperacillin, penicillin g, temocillin, ticarcillin, amoxicillin/clavulanate, ampicillin/sulbactam, piperacillin/tazobactam and/or ticarcillin/clavulanate); polypeptides (such as bacitracin, colistin, or polymyxin b); quinolones (such as, for example, ciprofloxacin, enoxacin, gatifloxacin, levofioxacin, lomefioxacin, moxifioxacin, nalidixic acid, norfloxacin, ofloxacin, trovafioxacin, grepafloxacin, sparfioxacin, or temafloxacin); sulfonamides (such as, for example, mafenide, sulfonamidochrysoidme, sulfacetamide, sulfadiazine, silver sulfadiazine, sulfamethizole, sulfamethoxazole, sulfanilimide, sulfasalazine, sulfisoxazole, or trimethoprim-sulfamethoxazole-co-trimoxazole); tetracyclines (such as, for example, demeclocycline, doxycycline, minocycline, oxytetracycline, or tetracycline); drugs against mycobacteria (such as, for example, clofazimine, dapsone, capreomycin, cycloserine, ethambutol, ethionamide, isoniazid, pyrazinamide, rifampicin, rifabutin, rifapentine, streptomycin); arsphenamine, chloramphenicol, fosfomycin, fusidic acid, linezolid, metronidazole, mupirocin, platensimycin, quinupristin/dalfopristin, rifaximin, thiamphenicol, tigecycline, imidazole, trimethoprim, or combinations thereof; or antifungal or antimycotic agents, such as azoles, echinocandins, polyenes, or combinations thereof.

In some embodiments, the at least one biologically active agent is embedded or impregnated into the material that forms the drug-eluting spacer.

In some embodiments, the at least one biologically active agent constitutes about 0.1% or less by weight, about 0.2% or less by weight, about 0.3% or less by weight, about 0.4% or less by weight, about 0.5% or less by weight, about 0.6% or less by weight, about 0.7% or less by weight, about 0.8% or less by weight, about 0.9% or less by weight, about 1.0% or less by weight, about 1.1% or less by weight, about 1.2% or less by weight, about 1.3% or less by weight, about 1.4% or less by weight, about 1.5% or less by weight, about 1.6% or less by weight, about 1.7% or less by weight, about 1.8% or less by weight, about 1.9% or less by weight, about 2.0% or less by weight, about 2.1% or less by weight, about 2.2% or less by weight, about 2.3% or less by weight, about 2.4% or less by weight, about 2.5% or less by weight, about 2.6% or less by weight, about 2.7% or less by weight, about 2.8% or less by weight, about 2.9% or less by weight, about 3.0% or less by weight, about 3.2% or less by weight, about 3.5% or less by weight, about 3.8% or less by weight, about 4.0% or less by weight, about 4.5% or less by weight, about 5.0% or less by weight, about 7.0% or less by weight, about 10.0% or less by weight, about 15.0% or less by weight, about 20.0% or less by weight, about 30.0% or less by weight, about 40.0% or less by weight, about 50.0% or less by weight of the total weight of the material that forms the drug-eluting spacer.

In some embodiments, the at least one biologically active agent is incorporated into the material used to fabricate the individual components of the spacer. In some embodiments, the at least one biologically active agent is homogeneously distributed throughout the material used to fabricate the components of the spacer. In some embodiments, all of the individual components have the at least one biologically active agent incorporated. In some embodiments, the at least one biologically active agent is incorporated throughout 100% of the volume of the material forming the component. Alternatively, the at least one biologically active agent may be incorporated into less than 100% of the volume of the material forming the component. For example, the at least one biologically active agent may be incorporated into 90%, 80%, 70%, 60%, 50%, 40%, 30%, 20%, 10%, 9%, 8%, 7%, 6%, 5%, 4%, 3%, 2%, or 1% of the volume of the material forming the component. In these instances, the at least one biologically active avent may be incorporated into a discrete region of the component. Non-limiting examples of discrete regions include, for example, regions of the component that contact bone, and the like.

In some embodiments, the at least one biological agent is coated onto the individual components that form a spacer according to some embodiments of the present invention.

In some embodiments, the spacer is impregnated with at least one antibiotic, but is not formulated for elution of the at least one antibiotic. In such embodiments, the association of the at least one antibiotic with the spacer prevents or reduces growth of a bacterium on or in the spacer, other surfaces of the spacer, or on a tissue that contacts the antibiotic-impregnated spacer or positioned in an area within which the antibiotic diffuses.

In some embodiments, a spacer is impregnated with an antibiotic formulated for elution of the antibiotic. In such embodiments, the association of the antibiotic, when implanted, prevents or reduces the growth of bacteria on or surrounding the spacer wherein the spacer does not need to come in direct contact with the bacteria because the antibiotic diffuses out of the spacer.

In some embodiments, the at least one biologically active agent is embedded or impregnated into the material that forms the drug-eluting spacer according to the methods described in International Patent Application Publication No. WO2013059745A1.

In some embodiments, the at least one biologically active agent is embedded or impregnated into the material that forms the drug-eluting spacer according to the methods described in U.S. Patent Application Publication No. 20150012105A1.

In some embodiments, the at least one biologically active agent is embedded or impregnated into the material that forms the drug-eluting spacer according to the methods described in U.S. Pat. No. 8,147,861.

### Spacer Material

In some embodiments, the material that forms the drug-eluting spacer is bone cement. In some embodiments, the bone cement includes methyl methacrylate. In some embodiments the bone cement includes a methyl methacrylate monomer. In some embodiments, the bone cement includes poly(methyl methacrylate) (“PMMA”).

In some embodiments, the material that forms the drug-eluting spacer is a biodegradable polymer. Exemplary polymeric materials include but are not limited to a biocompatible or bioabsorbable polymer that is one or more of poly(DL-lactide), poly(L-lactide), poly(L-lactide), poly(L-lactide-co-D,L-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof In some embodiments, the polymeric material comprises a combination of these polymers.

In some embodiments, the polymeric material comprises poly(D,L-lactide-co-glycolide). In some embodiments, the polymeric material comprises poly(D,L-lactide). In some embodiments, the polymeric material comprises poly(L-lactide).

Additional exemplary polymers include but are not limited to poly(D-lactide) (PDLA), polymandelide (PM), polyglycolide (PGA), poly(L-lactide-co-D,L-lactide) (PLDLA), poly(D,L-lactide) (PDLLA), poly(D,L-lactide-co-glycolide) (PLGA) and poly(L-lactide-co-glycolide) (PLLGA).

Additional examples of biocompatible biodegradable polymers include, without limitation, polycaprolactone, poly(L-lactide), poly(D,L-lactide), poly(D,L-lactide-co-PEG) block copolymers, poly(D,L-lactide-co-trimethylene carbonate), poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, polyalkylene oxalates, polyphosphazenes, PHA-PEG, and combinations thereof. The PHA may include poly(α-hydroxyacids), poly(β-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate), MED610, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof.

In some embodiments, the material that forms the drug-eluting spacer is selected from the polymeric materials described in International Patent Application Publication No. WO2013059745A1.

In some embodiments, the material that forms the drug-eluting spacer is selected from the materials described in U.S. Patent Application Publication No. 20150012105A1.

In some embodiments, the material that forms the drug-eluting spacer is selected from the materials described in U.S. Pat. No. 8,147,861.

In some embodiments, the drug-eluting spacer supports dynamic loads, and allows the articular function of the joint to be maintained.

### Joints Treated According to Some Embodiments of the Present Invention

Any joint is suitable to be treated with a drug-eluting spacer according to some embodiments of the present invention. Such joints include for example, knee joints, shoulder joints, hip joints, and the like.

In some embodiments, the spacer is implanted, and remains implanted for a time sufficient to treat the infection. After such time, the spacer is removed.

For example, the drug-eluting spacer may be utilized to treat an infection of a shoulder joint. In these embodiments, the drug-eluting spacer may be fabricated to resemble a shoulder implant system, such as the system disclosed in U.S. Pat. No. 8,241,366.

In another example, the drug-eluting spacer may be utilized to treat an infection of a hip joint. In these embodiments, the drug-eluting spacer may be fabricated to resemble a hip implant system, such as the system disclosed in U.S. Pat. No. 6,911,048.

In some embodiments, the drug-eluting spacer is configured for a knee joint. Thus, in some embodiments, the present invention provides a drug-eluting spacer for temporary implantation in a knee joint of a patient,

wherein the drug-eluting spacer is configured to elute at least one biologically active agent in an amount effective to treat an infection of the knee joint of the patient,

wherein the drug-eluting spacer comprises:
a) a femoral component configured to interface with a femur of the patient;
b) a tibial tray component, wherein the tibial tray comprises an upper surface and a lower surface, and the lower surface of the tibial tray component is disposed adjacent a tibia of the patient
   wherein the lower surface of the tibial tray component comprises a shaft extending downward from the lower surface, and
   wherein the shaft is adapted to be located axially within the tibia; and
c) a tibial insert component, wherein the tibial insert component comprises an upper surface and a lower surface;
   wherein the upper and lower surfaces are separated by a thickness,
   wherein the lower surface of the tibial insert component is configured to lockingly engage with the upper surface of the tibial tray component,
   wherein the locked tibial insert component and tibial tray component carry joint loads when implanted in the patient, and
   wherein the upper surface of the tibial insert component is configured to receive the femoral component in an articulating manner.

In some embodiments, the femoral component has an anterior side and a posterior side, the femoral component including a pair of laterally spaced condylar portions, each of which has a surface which is configured to match generally the lateral profile of an anatomical femoral condyle.

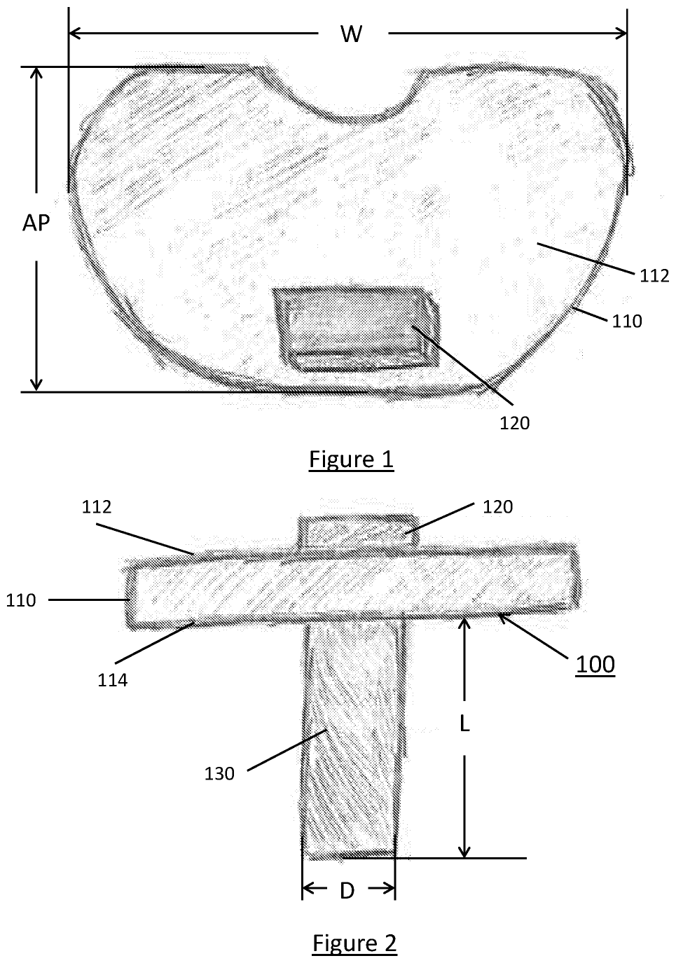

An exemplary embodiment of drug-eluting spacer configured for a knee joint is shown in FIGS. 1-10. FIG. 1 shows a top view of a tibial tray component 100 of an exemplary drug-eluting spacer. In some embodiments, the tibial tray component 100 is configured to be temporarily implanted adjacent to a resected proximal end of a tibia of a patient. In some embodiments, the tibial tray component includes a body 110 having a superior surface 112 and a projection 120 extending from the superior surface 112. In some embodiments, the tibial tray component 100 has a width W in the medial-lateral direction and a length AP in the anterior-posterior direction. In some embodiments, the tibial tray component 100 may be fabricated in a variety of nominal sizes (e.g., small, medium, large, extra-large). In some embodiments, a small size of the tibial tray component 100 has a width W in a range of between 50 mm and 60 mm and a length AP in a range of between 25 mm and 33 mm. In some embodiments, a medium size of the tibial tray component 100 has a width W in a range of between 60 mm and 70 mm and a length AP in a range of between 34 mm and 44 mm. In some embodiments, a large size of the tibial tray component 100 has a width W in a range of between 70 mm and 80 mm and a length AP in a range of between 45 mm and 53 mm. In some embodiments, an extra-large size of the tibial tray component 100 has a width W in a range of between 90 mm and 100 mm and a length AP in a range of between 54 mm and 62 mm.

In some embodiments, the tibial tray component 100 is formed from a material that is any of the materials listed above. In some embodiments, the tibial tray component 100 is formed from a combination of more than one of the materials listed above. In some embodiments, at least one biologically active agent is included in (e.g., embedded in, impregnated into, coated onto, etc.) the material that forms the tibial tray component 100. In some embodiments, the at least one biologically active agent includes a quantity of the at least one biologically active agent that is between 2.5% and 8% of the material that forms the tibial tray component 100 by weight. In some embodiments, the at least one biologically active agent includes a quantity of the at least one biologically active agent that is between 2.5% and 20% of the material that forms the tibial tray component 100 by weight. In some embodiments, the at least one biologically active agent includes gentamicin. In some embodiments, the at least one biologically active agent includes a quantity of gentamicin that is between 2.5% and 8% of the material that forms the tibial tray component 100 by weight. In some embodiments, the at least one biologically active agent includes a quantity of gentamicin that is between 2.5% and 20% of the material that forms the tibial tray component 100 by weight. In some embodiments, the at least one biologically active agent includes vancomycin. In some embodiments, the at least one biologically active agent includes a quantity of vancomycin that is between 2.5% and 8% of the material that forms the tibial tray component 100 by weight. In some embodiments, the at least one biologically active agent includes a quantity of vancomycin that is between 2.5% and 20% of the material that forms the tibial tray component 100 by weight. In some embodiments, the at least one biologically active agent includes gentamicin and vancomycin. In some embodiments, the at least one biologically active agent includes a quantity of gentamicin that is between 2.5% and 4% of the material that forms the tibial tray component 100 by weight and a quantity of vancomycin that is between 2.5% and 4% of the material that forms the tibial tray component 100 by weight. In some embodiments, the at least one biologically active agent includes a quantity of gentamicin that is between 2.5% and 20% of the material that forms the tibial tray component 100 by weight and a quantity of vancomycin that is between 2.5% and 20% of the material that forms the tibial tray component 100 by weight.

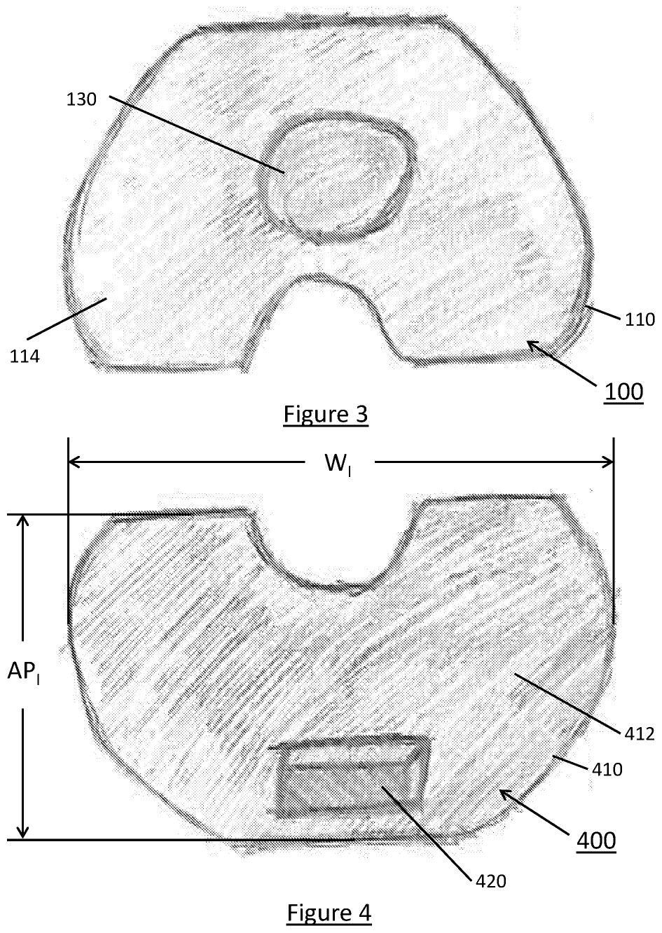

FIG. 2 shows an anterior/posterior view of the tibial tray component 100 shown in FIG. 1. As shown in FIG. 2, the body 110 of the tibial tray component 100 has an inferior surface 114 opposite the superior surface 112. In some embodiments, the tibial tray component 100 includes a shaft 130 extending from the inferior surface 114 of the body 110. In some embodiments, the shaft 130 is configured to be positioned axially within a tibia of a patient. In some embodiments, the shaft 130 has a diameter D and a length L. As noted above, in some embodiments, the tibial tray component 100 may be fabricated in a variety of nominal sizes (e.g., small, medium, large, extra-large). In some embodiments, a small size of the tibial tray component 100 includes a shaft 130 having a diameter D in a range of between 5 mm and 10 mm and a length L in a range of between 5 mm and 25 mm. In some embodiments, a medium size of the tibial tray component 100 includes a shaft 130 having a diameter D in a range of between 10 mm and 15 mm and a length L in a range of between 25 mm and 50 mm. In some embodiments, a large size of the tibial tray component 100 includes a shaft 130 having a diameter D in a range of between 15 mm and 20 mm and a length L in a range of between 50 mm and 100 mm. In some embodiments, an extra large size of the tibial tray component 100 includes a shaft 130 having a diameter D in a range of between 20 mm and 25 mm and a length L in a range of between 100 mm and 175 mm. FIG. 3 shows a bottom view of the tibial tray component 100 shown in FIGS. 1 and 2.

FIG. 4 shows a bottom view of a tibial insert 400. In some embodiments, the tibial insert 400 includes a body 410 having an inferior surface 412. In some embodiments, a recess 420 is formed in the inferior surface 412. In some embodiments, the recess 420 is configured to receive the projection 120 of the tibial tray component 100 so as to retain the tibial tray component 100 and the tibial insert 400 in proximity to one another, with the inferior surface 412 of the tibial insert 400 abutting the superior surface 112 of the tibial tray component 100. In some embodiments, the tibial insert 400 has a width W_{I} in the medial-lateral direction and a length AP_{I} in the anterior-posterior direction. In some embodiments, the tibial insert 400 may be fabricated in a variety of nominal sizes (e.g., small, medium, large, extra-large). In some embodiments, various nominal sizes of the tibial insert 400 may be sized and shaped so as to have a profile similar to that of ones of the tibial tray component 100 having the same nominal size. In some embodiments, a small size of the tibial insert 400 has a width W_{I} in a range of between 50 mm and 70 mm and a length AP_{I} in a range of between 30 mm and 42 mm. In some embodiments, a medium size of the tibial insert 400 has a width W_{I} in a range of between 60 mm and 80 mm and a length AP_{I} in a range of between 36 mm and 48 mm. In some embodiments, a large size of the tibial insert 400 has a width W_{I} in a range of between 70 mm and 90 mm and a length AP_{I} in a range of between 42 mm and 54 mm. In some embodiments, an extra-large size of the tibial insert 400 has a width W_{I} in a range of between 80 mm and 100 mm and a length AP_{I} in a range of between 48 mm and 60 mm.

In some embodiments, the tibial insert 400 is formed from a material that is any of the materials listed above. In some embodiments, the tibial insert 400 is formed from a combination of more than one of the materials listed above. In some embodiments, at least one biologically active agent is included in (e.g., embedded in, impregnated into, etc.) the material that forms the tibial insert 400. In some embodiments, the at least one biologically active agent includes a quantity of the at least one biologically active agent that is between 2.5% and 8% of the material that forms the tibial insert 400 by weight. In some embodiments, the at least one biologically active agent includes a quantity of the at least one biologically active agent that is between 2.5% and 20% of the material that forms the tibial insert 400 by weight. In some embodiments, the at least one biologically active agent includes gentamicin. In some embodiments, the at least one biologically active agent includes a quantity of gentamicin that is between 2.5% and 8% of the material that forms the tibial insert 400 by weight. In some embodiments, the at least one biologically active agent includes a quantity of gentamicin that is between 2.5% and 20% of the material that forms the tibial insert 400 by weight. In some embodiments, the at least one biologically active agent includes vancomycin. In some embodiments, the at least one biologically active agent includes a quantity of vancomycin that is between 2.5% and 8% of the material that forms the tibial insert 400 by weight. In some embodiments, the at least one biologically active agent includes a quantity of vancomycin that is between 2.5% and 20% of the material that forms the tibial insert 400 by weight. In some embodiments, the at least one biologically active agent includes gentamicin and vancomycin. In some embodiments, the at least one biologically active agent includes a quantity of gentamicin that is between 2.5% and 4% of the material that forms the tibial insert 400 by weight and a quantity of vancomycin that is between 2.5% and 4% of the material that forms the tibial insert 400 by weight. In some embodiments, the at least one biologically active agent includes a quantity of gentamicin that is between 2.5% and 20% of the material that forms the tibial insert 400 by weight and a quantity of vancomycin that is between 2.5% and 20% of the material that forms the tibial insert 400 by weight.

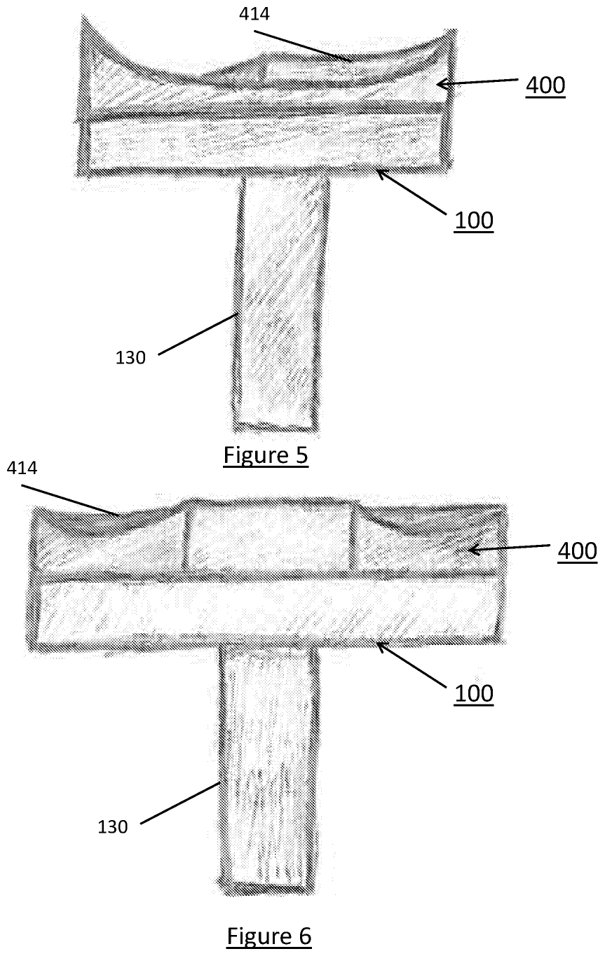

FIG. 5 shows a medial-lateral view of the tibial insert 400 assembled with the tibial tray component 100. In some embodiments, the body 410 of the tibial insert 400 has a superior surface 414 opposite the inferior surface 412 of the body 410. In some embodiments, the superior surface 414 is contoured so as to engage a femoral component. FIG. 6 shows an anterior-posterior view of the tibial insert 400 and the tibial tray component 100 shown in FIG. 5.

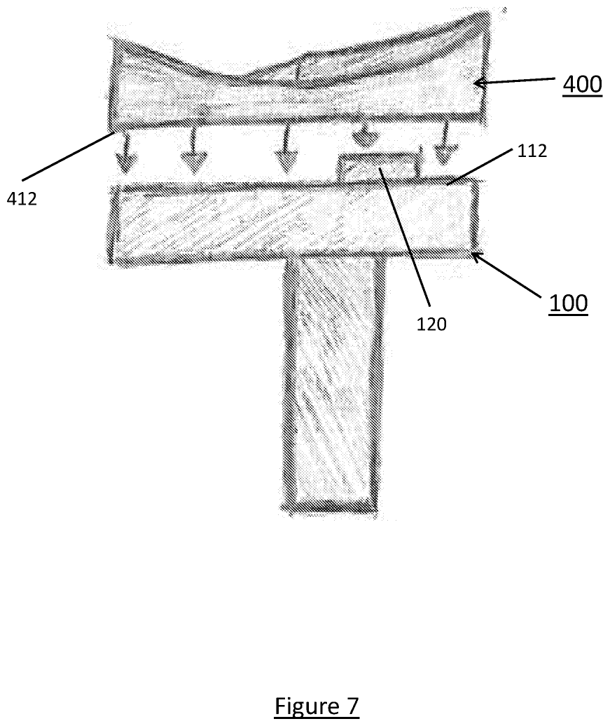

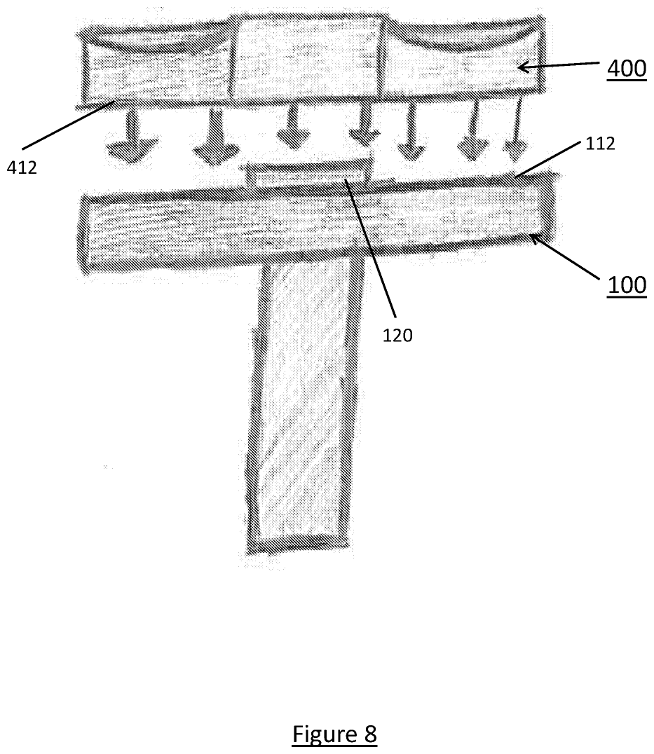

FIG. 7 shows a medial-lateral view of assembly of the tibial insert 400 and the tibial tray component 100 shown in FIG. 5, with arrows showing the direction of movement as the tibial insert 400 is brought into proximity with the tibial tray component 100. FIG. 8 shows an anterior-posterior view of the assembly process shown in FIG. 7.

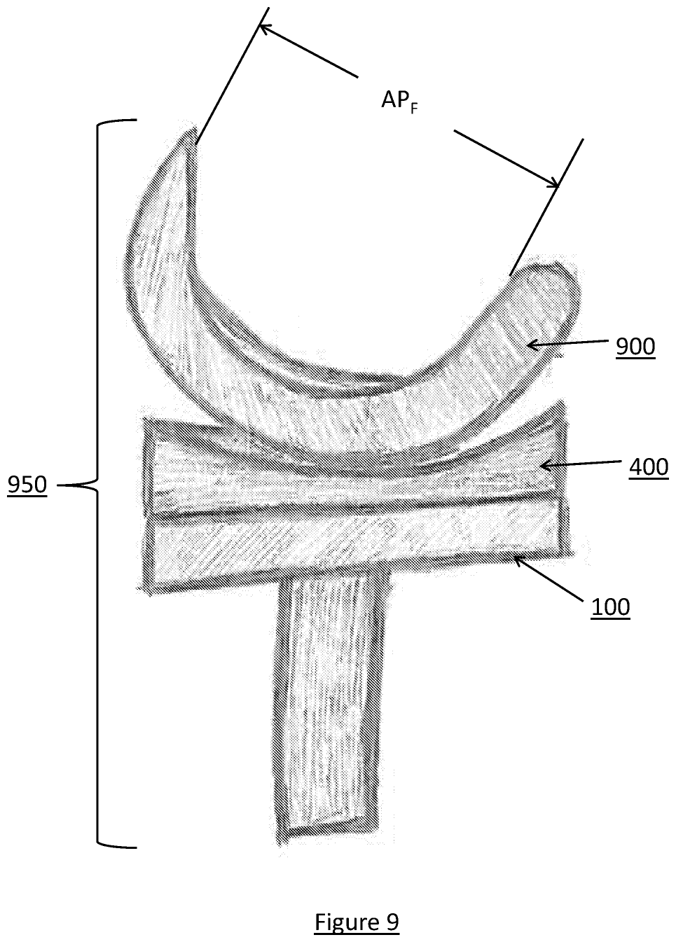

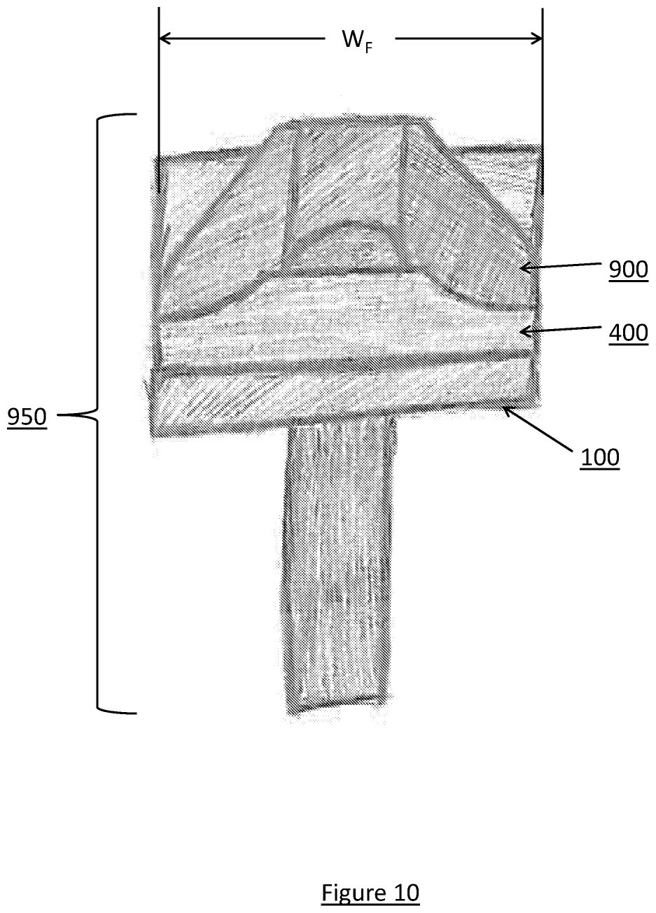

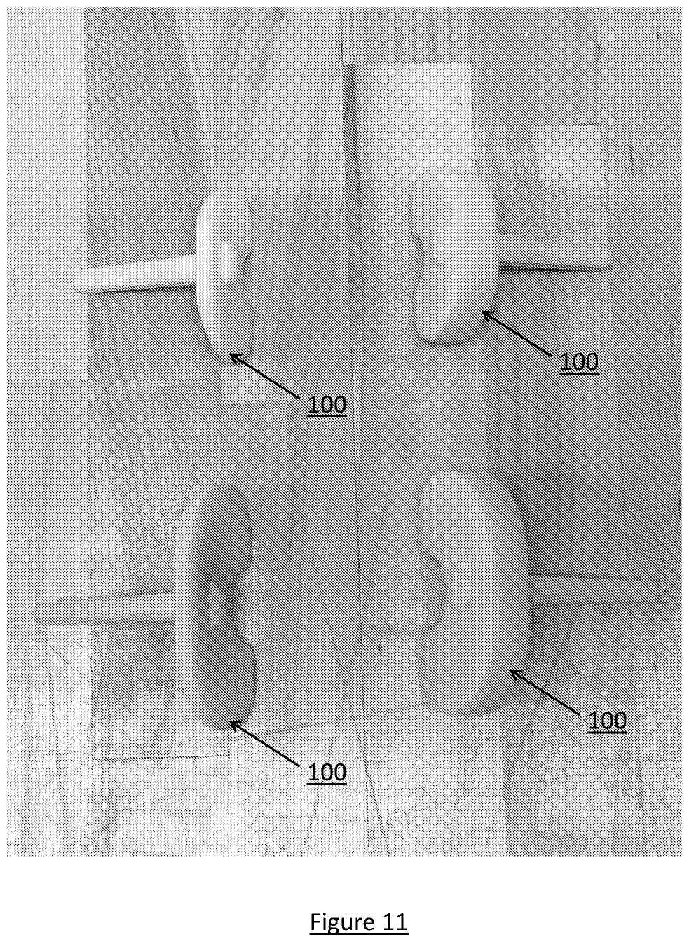

FIG. 9 shows a medial-lateral view of the tibial insert 400 and tibial tray component 100 shown in FIG. 5, further assembled with a femoral component 900 to form a spacer assembly 950. In some embodiments, the femoral component 900 is configured to be temporarily implanted adjacent to a resected end of a femur of a patient. In some embodiments, the femoral component 900 is configured to abut the tibial insert 400 so as to provide a temporary replacement for a knee joint of a patient. In some embodiments, the femoral component 900 has a width W_{F} in the medial-lateral direction and an internal length AP_{F} in the anterior-posterior direction. In some embodiments, the femoral component 900 may be fabricated in a variety of nominal sizes (e.g., small, medium, large, extra-large). In some embodiments, various nominal sizes of the femoral component 900 may be sized and shaped so as to engage ones of the tibial insert 400 having the same nominal size. In some embodiments, a small size of the femoral component 900 has a width W_{F} in a range of between 44 mm and 64 mm and a length AP_{F} in a range of between 33 mm and 47 mm. In some embodiments, a medium size of the femoral component 900 has a width W_{F} in a range of between 54 mm and 74 mm and a length AP_{F} in a range of between 40 mm and 54 mm. In some embodiments, a large size of the femoral component 900 has a width W_{F} in a range of between 64 mm and 84 mm and a length AP_{F} in a range of between 47 mm and 61 mm. In some embodiments, an extra-large size of the femoral component 900 has a width W_{F} in a range of between 74 mm and 94 mm and a length AP_{F} in a range of between 54 mm and 68 mm. FIG. 10 shows an anterior-posterior view of the spacer assembly 950 shown in FIG. 9.

In some embodiments, the femoral component 900 is formed from a material that is any of the materials listed above. In some embodiments, the femoral component 900 is formed from a combination of more than one of the materials listed above. In some embodiments, at least one biologically active agent is included in (e.g., embedded in, impregnated into, etc.) the material that forms the femoral component 900. In some embodiments, the at least one biologically active agent includes a quantity of the at least one biologically active agent that is between 2.5% and 8% of the material that forms the femoral component 900 by weight. In some embodiments, the at least one biologically active agent includes a quantity of the at least one biologically active agent that is between 2.5% and 20% of the material that forms the femoral component 900 by weight. In some embodiments, the at least one biologically active agent includes gentamicin. In some embodiments, the at least one biologically active agent includes a quantity of gentamicin that is between 2.5% and 8% of the material that forms the femoral component 900 by weight. In some embodiments, the at least one biologically active agent includes a quantity of gentamicin that is between 2.5% and 20% of the material that forms the femoral component 900 by weight. In some embodiments, the at least one biologically active agent includes vancomycin. In some embodiments, the at least one biologically active agent includes a quantity of vancomycin that is between 2.5% and 8% of the material that forms the femoral component 900 by weight. In some embodiments, the at least one biologically active agent includes a quantity of vancomycin that is between 2.5% and 20% of the material that forms the femoral component 900 by weight. In some embodiments, the at least one biologically active agent includes gentamicin and vancomycin. In some embodiments, the at least one biologically active agent includes a quantity of gentamicin that is between 2.5% and 4% of the material that forms the femoral component 900 by weight and a quantity of vancomycin that is between 2.5% and 4% of the material that forms the femoral component 900 by weight. In some embodiments, the at least one biologically active agent includes a quantity of gentamicin that is between 2.5% and 20% of the material that forms the femoral component 900 by weight and a quantity of vancomycin that is between 2.5% and 20% of the material that forms the femoral component 900 by weight.

In some embodiments, the femoral component of the drug-eluting spacer is fabricated to resemble the femoral component disclosed in U.S. Pat. No. 6,730,128.

In some embodiments, the tibial tray component of the drug-eluting spacer is fabricated to resemble the tibial tray component disclosed in U.S. Pat. No. 6,730,128.

In some embodiments, the tibial tray component and the tibial spacer component are fabricated to resemble the tibial tray and spacers disclosed in U.S. Pat. No. 5,702,464.

Without intending to be limited to any particular theory, one goal of knee arthroplasty is to function as a normal knee and in this regard the following two parameters to achieve this goal include: appropriate dimensioning of the prosthesis (i.e., having the prosthesis match the morphology of the patient's knee); and appropriate orientation of the prosthesis (e.g., having the centerline of the prosthesis replicate the anatomical centerline).

With regard to dimensioning, one parameter is coverage of the knee. For the purposes of describing and claiming the present invention, the term “size” is intended to refer to the overall dimension of the tibial insert in the transverse plane.

Further, and again with regard to dimensioning, another parameter is thickness, wherein a tibial insert component is selected, having a thickness that appropriately adjusts the gap between the femur and the tibia). For the purposes of describing and claiming the present invention, the term “thickness” is intended to refer to the height of the tibial insert component measured between: (a) the lower surface surface configured to configured to lockingly engage with the upper surface of the tibial tray component; and (b) a low point on the upper (i.e. articular) surface of the tibial insert component. Another parameter for dimensioning is the curvature of the upper (articular surfaces) if the tibial insert component.

Accordingly, in order to achieve the desired coverage and gap between the femur and the tibia, a surgeon is required to select a tibial insert component from a plurality of individual tibial insert components of varying sizes, thicknesses, and curvature of the upper surface.

In some embodiments, the drug-eluting spacer further comprises a tibial spacer component, positionable between the tibial tray component and the tibial insert component, wherein the tibial spacer component comprises an upper surface and a lower surface, wherein the upper surface of the tibial spacer component is lockingly engaged with the lower surface of the tibial insert component, and wherein the lower surface of the tibial spacer component is lockingly engaged with the upper surface of the tibial tray component.

In one embodiment, the locked tibial insert component/tibial spacer component and tibial tray component carry joint loads when implanted in the patient.

Exemplar tibial spacer components suitable for use in the device according to some embodiments of the present invention are disclosed in U.S. Patent Application Publication No. 2008/0051908 A1.

Accordingly, in some embodiments, the present invention provides kit to form a drug-eluting spacer for temporary implantation in a knee joint of a patient,

wherein the drug-eluting spacer is configured to elute at least one biologically active agent in an amount effective to treat an infection of the knee joint of the patient,

wherein the kit comprises:
a) a femoral component configured to interface with a femur of the patient;
b) a tibial tray component, wherein the tibial tray component comprises an upper surface and a lower surface, and the lower surface of the tibial tray component is disposed adjacent a tibia of the patient
   wherein the lower surface of the tibial tray component comprises a shaft extending downward from the lower surface, and
   wherein the shaft is adapted to be located axially within the tibia;
c) a plurality of tibial insert components of a first size, wherein each individual tibial insert component within the plurality comprises an upper surface and a lower surface;
   wherein the upper and lower surfaces are separated by a thickness,
   wherein each individual tibial insert component within the plurality has a different thickness from any other individual tibial insert components within the plurality,
   wherein the lower surface of the tibial insert component is configured to lockingly engage with the upper surface of the tibial tray component,
   wherein the locked tibial spacer component and tibial tray component carry joint loads when implanted in the patient, and
   wherein the upper surface of the tibial insert component is configured to receive the femoral component in an articulating manner; and
d) a plurality of tibial insert components of a at least one additional size, wherein each individual tibial insert component within the plurality comprises an upper surface and a lower surface;
   wherein the upper and lower surfaces are separated by a thickness,
   wherein each individual tibial insert component within the plurality has a different thickness from any other individual tibial insert components within the plurality,
   wherein the lower surface of the tibial insert component is configured to lockingly engage with the upper surface of the tibial tray component,
   wherein the locked tibial spacer component and tibial tray component carry joint loads when implanted in the patient, and
   wherein the upper surface of the tibial insert component is configured to receive the femoral component in an articulating manner.

In some embodiments, the drug-eluting spacer further comprises a tibial spacer component, positionable between the tibial tray component and the tibial insert component, wherein the tibial spacer component comprises an upper surface and a lower surface, wherein the upper surface of the tibial spacer component is lockingly engaged with the lower surface of the tibial insert component, and wherein the lower surface of the tibial spacer component is lockingly engaged with the upper surface of the tibial tray component.

In some embodiments, the locked tibial insert component/tibial spacer component and tibial tray component carry joint loads when implanted in the patient.

In some embodiments, the size of the tibial inserts of the first size is the same as the size of the tibial tray.

In some embodiments, the size of the tibial inserts of the at least one additional size are larger than the size of the tibial inserts of the first size. Alternatively, the size of the tibial inserts of the at least one additional size are smaller than the size of the tibial inserts of the first size.

Exemplary tibial insert components of the first size and the at least one additional size, suitable for use in a device according to some embodiments of the present invention are disclosed in U.S. Pat. No. 8,414,653.

In some embodiments, the femoral component has an anterior side and a posterior side, the femoral component including a pair of laterally spaced condylar portions, each of which has a surface which is configured to match generally the lateral profile of an anatomical femoral condyle.

Publications cited throughout this document are hereby incorporated by reference in their entirety.

1-20. (canceled)

21. A kit to form a drug-eluting spacer for temporary implantation in a knee joint of a patient, kit comprising:
a femoral component configured to interface with a femur of the patient;
a tibial tray component having an upper surface, a lower surface opposite the upper surface, and a shaft extending from the lower surface, the shaft configured to be positioned axially within a tibia of the patient, the lower surface configured to interface with the tibia of the patient;
a first plurality of tibial insert components of a first size, each of the first plurality of tibial insert components having an upper surface, a lower surface opposite the upper surface, and a thickness between the upper and lower surfaces, each of the tibial insert components in the first plurality having a different thickness from any other individual tibial insert component within the first plurality of tibial insert components, the lower surface of each of the tibial insert components within the first plurality configured to lockingly engage the upper surface of the tibial tray component, the upper surface of each of the tibial insert components within the first plurality configured to receive the femoral component in an articulating manner; and
a second plurality of tibial insert components of a second size, each of the second plurality of tibial insert components having an upper surface, a lower surface opposite the upper surface, and a thickness between the upper and lower surfaces, each of the tibial insert components in the second plurality having a different thickness from any other individual tibial insert component within the second plurality of tibial insert components, the lower surface of each of the tibial insert components within the second plurality configured to lockingly engage the upper surface of the tibial tray component, the upper surface of each of the tibial insert components within the second plurality configured to receive the femoral component in an articulating manner,
wherein the femoral component, the tibial tray component, and each of the tibial insert components are made from a spacer material including a structural material and at least one biologically active agent,
wherein the structural material includes at least one of bone cement, a polymer, a biodegradable polymer, a biocompatible polymer, a bioabsorbable polymer, or combinations thereof, and
wherein a drug-eluting spacer formed from the femoral component, the tibial tray component, and a selected one of the tibial insert components carries joint loads when implanted in the patient, and
wherein the drug-eluting spacer is configured to elute the at least one biologically active agent in an amount effective to treat an infection of the knee joint of the patient.

22. The kit of claim 21, wherein the at least one biologically active agent includes at least one antibiotic.

23. The kit of claim 22, wherein the at least one antibiotic includes at least one of an aminoglycoside, an ansamycin, a carbapenem, a cephalosporin, a glycopeptide, a lincosamide, a macrolide, a monobactam, a penicillin, a penicillin combination, a polypeptide, a quinolone, a sulfonamide, a tetracycline, a drug against mycobacteria, arsphenamine, chloramphenicol, fosfomycin, fusidic acid, linezolid, metronidazole, mupirocin, platensimycin, quinupristin/dalfopristin, rifaximin, thiamphenicol, tigecycline, imidazole, trimethoprim, or combinations thereof

24. The kit of claim 22, wherein the at least one antibiotic includes at least one of vancomycin, gentamicin, or combinations thereof

25. The kit of claim 24, wherein the at least one antibiotic includes at least one of vancomycin at a concentration of between 2.5% and 20% by weight, gentamicin at a concentration of between 2.5% and 20% by weight, or combinations thereof.

26. The kit of claim 21, wherein the at least one biologically active agent includes at least one antifungal agent.

27. The kit of claim 26, wherein the at least one antifungal agent includes at least one of an azole, an echinocandin, a polyene, or combinations thereof

28. The kit of claim 23, wherein the at least one biologically active agent comprises about 20% or less of the spacer material by weight.

29. The kit of claim 21, wherein the at least one biologically active agent is at least one of embedded into the structural material, impregnated into the structural material, or coated onto the structural material.

30. The kit of claim 21, wherein the tibial tray component includes a projection projecting from the upper surface thereof, wherein each of the plurality of tibial insert components includes a recess formed within the lower surface thereof, and wherein the projection of the tibial tray component and the recess of a selected one of the tibial insert components are configured to cooperate to lockingly engage the selected one of the tibial insert components to the tibial tray component when the lower surface of the selected one of the tibial insert components abuts the upper surface of the tibial tray component.

31. The kit of claim 21, wherein the shaft of the tibial tray component has a diameter in a range between 5 mm and 25 mm and a length in a range between 5 mm and 175 mm.

32. The kit of claim 21, wherein the first size is the same as a size of the tibial tray.

33. The kit of claim 32, wherein the second size is larger than the first size.

34. The kit of claim 32, wherein the second size is smaller than the first size.

35. A method, comprising:
providing a kit, the kit including:
a femoral component configured to interface with a femur of the patient;
a tibial tray component having an upper surface, a lower surface opposite the upper surface, and a shaft extending from the lower surface, the shaft configured to be positioned axially within a tibia of the patient, the lower surface configured to interface with the tibia of the patient;
a first plurality of tibial insert components of a first size, each of the first plurality of tibial insert components having an upper surface, a lower surface opposite the upper surface, and a thickness between the upper and lower surfaces, each of the tibial insert components in the first plurality having a different thickness from any other individual tibial insert component within the first plurality of tibial insert components, the lower surface of each of the tibial insert components within the first plurality configured to lockingly engage the upper surface of the tibial tray component, the upper surface of each of the tibial insert components within the first plurality configured to receive the femoral component in an articulating manner; and
a second plurality of tibial insert components of a second size, each of the second plurality of tibial insert components having an upper surface, a lower surface opposite the upper surface, and a thickness between the upper and lower surfaces, each of the tibial insert components in the second plurality having a different thickness from any other individual tibial insert component within the second plurality of tibial insert components, the lower surface of each of the tibial insert components within the second plurality configured to lockingly engage the upper surface of the tibial tray component, the upper surface of each of the tibial insert components within the second plurality configured to receive the femoral component in an articulating manner, wherein the femoral component, the tibial tray component, and each of the tibial insert components are made from a spacer material including a structural material and at least one biologically active agent,
wherein the structural material includes at least one of bone cement, a polymer, a biodegradable polymer, a biocompatible polymer, a bioabsorbable polymer, or combinations thereof, and
selecting a selected tibial insert component, wherein the selected tibial insert component is selected from the first plurality of tibial insert components or from the second plurality of tibial insert components;
assembling the tibial tray component, the selected tibial insert component, and the femoral component to produce a drug-eluting tibial spacer;
temporarily implanting the drug-eluting tibial spacer within a resected proximal tibia of a knee joint of a patient, whereby the drug-eluting tibial spacer is configured to elute the at least one biologically active agent in an amount effective to treat an infection in the knee joint of the patient; and
removing the drug-eluting tibial spacer from the resected proximal tibia of the knee joint of the patient,
wherein the step of removing the drug-eluting tibial spacer is performed after a time sufficient to treat the infection in the knee joint of the patient.

36. The method of claim 35, wherein the at least one biologically active agent is at least one of embedded into the structural material, impregnated into the structural material, or coated onto the structural material.

37. The method of claim 35, wherein the tibial tray component includes a projection projecting from the upper surface thereof, wherein each of the plurality of tibial insert components includes a recess formed within the lower surface thereof, and wherein the projection of the tibial tray component and the recess of a selected one of the tibial insert components are configured to cooperate to lockingly engage the selected one of the tibial insert components to the tibial tray component when the lower surface of the selected one of the tibial insert components abuts the upper surface of the tibial tray component.

38. The method of claim 35, wherein the at least one biologically active agent includes at least one antibiotic.

39. The method of claim 38, wherein the at least one antibiotic includes at least one of an aminoglycoside, an ansamycin, a carbapenem, a cephalosporin, a glycopeptide, a lincosamide, a macrolide, a monobactam, a penicillin, a penicillin combination, a polypeptide, a quinolone, a sulfonamide, a tetracycline, a drug against mycobacteria, arsphenamine, chloramphenicol, fosfomycin, fusidic acid, linezolid, metronidazole, mupirocin, platensimycin, quinupristin/dalfopristin, rifaximin, thiamphenicol, tigecycline, imidazole, trimethoprim, vancomycin, gentamicin, or combinations thereof

40. The method of claim 35, wherein the at least one biologically active agent includes at least one antifungal agent.
